(19) United States
(12) Patent Application Publication
(10) Pub. No.: US 2011/0224501 A1
(43) Pub. Date: Sep. 15, 2011

(54) IN-HOME HEALTH MONITORING APPARATUS AND SYSTEM

(76) Inventor: Scotte Hudsmith, Brentwood, TN (US)

(21) Appl. No.: 13/046,707

(22) Filed: Mar. 12, 2011

Related U.S. Application Data
(60) Provisional application No. 61/313,407, filed on Mar. 12, 2010.

(51) Int. Cl. A61B 5/00

(52) U.S. Cl. 600/300

(57) ABSTRACT

A health monitoring system that allows users to maintain an autonomous lifestyle while providing health observation and reporting for family members, caregivers, and healthcare professionals. The system is supported by a network for critical, round-the-clock health monitoring and remote care, and may be used in the home or elsewhere. In one exemplary embodiment, the system comprises a touch-screen computing device that also may function as a television and video or DVD player, and provides a series of service options for users. The computing device is connected to a network and/or the Internet.

This application claims benefit of and priority to U.S. Provisional Application No. 61/313,407, filed Mar. 12, 2010, by Scotte Hudsmith, and is entitled to that filing date for priority. The specification, figures and complete disclosure of U.S. Provisional Application No. 61/313,407 are incorporated herein by specific reference for all purposes.

### FIELD OF INVENTION

This invention relates to an apparatus and system for health observation and reporting of individuals in a home.

### BACKGROUND OF THE INVENTION

As the population ages, the problems associated with providing appropriate care to seniors continue to increase. This is especially so for people are capable of living independently in their homes, and who do not want to leave their homes, but who also need regular health monitoring. They do not want to move in with family members, which may require moving to a different city, state or area of the country, and hiring a private nurse or day companion can be cost prohibitive.

Accordingly, what is needed is a system that allows people to remain living in their homes as they age, but receive cost-effective assistance and health monitoring services.

### SUMMARY OF THE INVENTION

In various embodiments, the present invention comprises a health monitoring system that allows users to maintain an autonomous lifestyle while providing health observation and reporting for family members, caregivers, and healthcare professionals. The apparatus and system is supported by a network for critical, round-the-clock health monitoring and remote care, and may be used in the home or elsewhere.

In one exemplary embodiment, the system comprises a touch-screen computing device. The computing device also may accept input from other sources, such as a keyboard, mouse, microphone, or the like. Thus, for example, a text message may be typed using a keyboard, voice recognition software, or a virtual keyboard presented on the touchscreen itself. The computing device also may function as a television and video or DVD player. The computing device is connected to a network and/or the Internet.

### DESCRIPTION OF THE DRAWINGS

FIG. 1 shows a view of a system dashboard menu in accordance with an embodiment of the present invention.

FIG. 2 shows another view of a system dashboard menu in accordance with an embodiment of the present invention.

FIGS. 3 through 13 show exemplary screenshots of the system of FIG. 2.

FIG. 14 shows an example of a social web site accessible through the system of FIG. 2.

FIG. 15 is a diagram of a system in accordance with an embodiment of the present invention.

FIGS. 16-23 show exemplary screenshots of a call center web application in accordance with an embodiment of the present invention.

FIG. 24-27 show exemplary screenshots of a family web application in accordance with an embodiment of the present invention.

### DETAILED DESCRIPTION OF EXEMPLARY EMBODIMENTS

In one exemplary embodiment the present invention comprises a health monitoring system that allows users to maintain an autonomous lifestyle while providing health observation and reporting for family members, caregivers, and healthcare professionals. A user may be a senior citizen, an individual living by themselves, or any other person or persons who need regular health monitoring services. The invention may be used in the home, residence or elsewhere. The apparatus and system is supported by a network for critical, round-the-clock health monitoring and remote care.

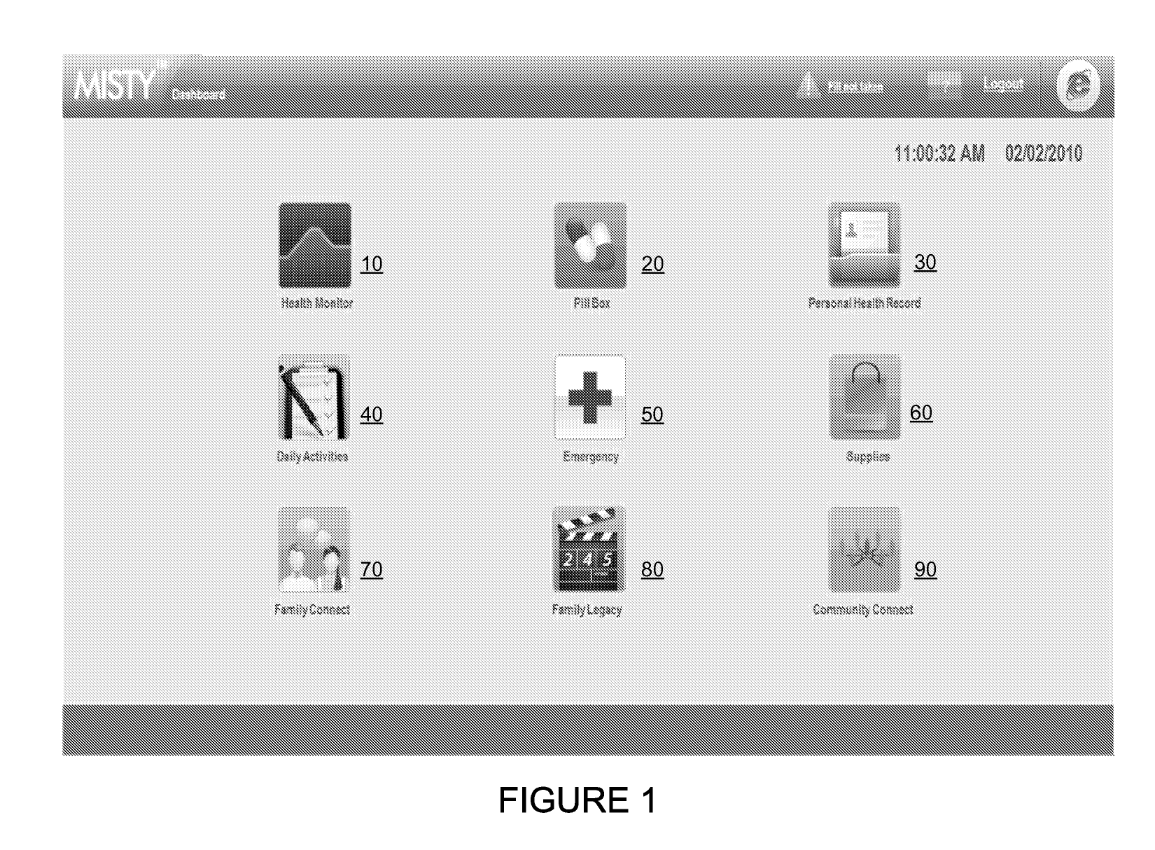

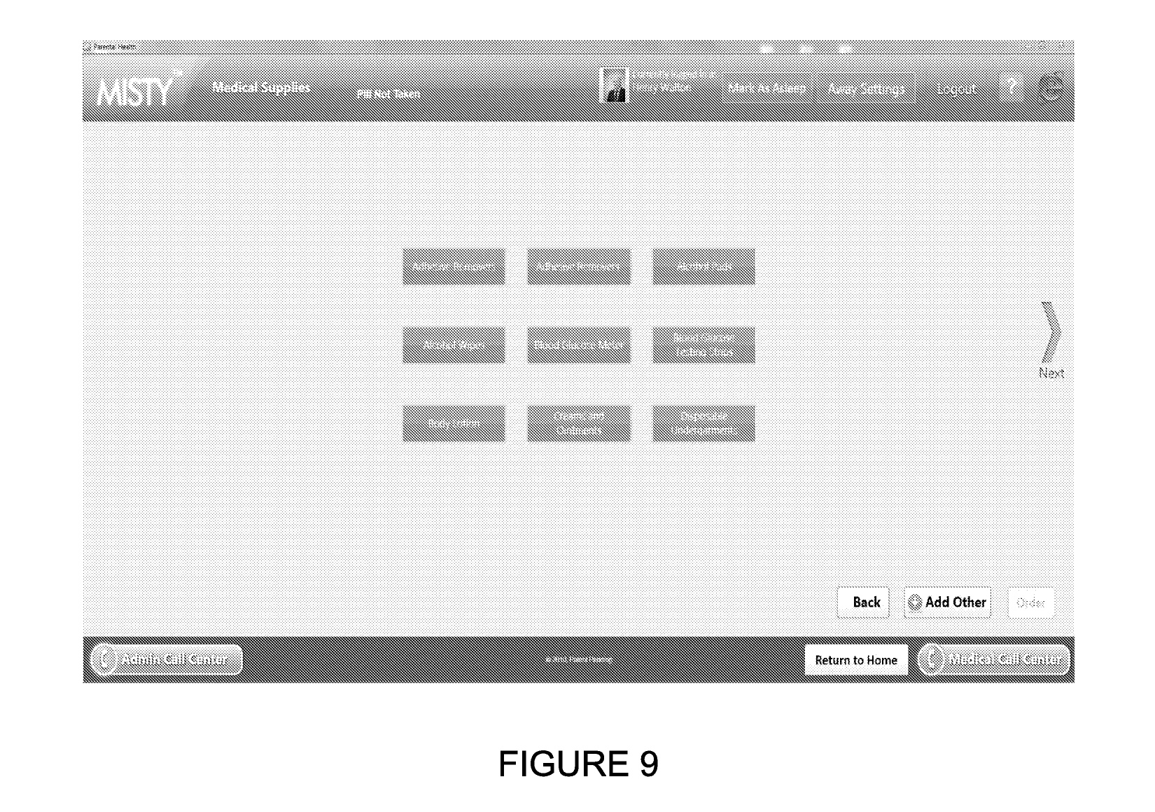

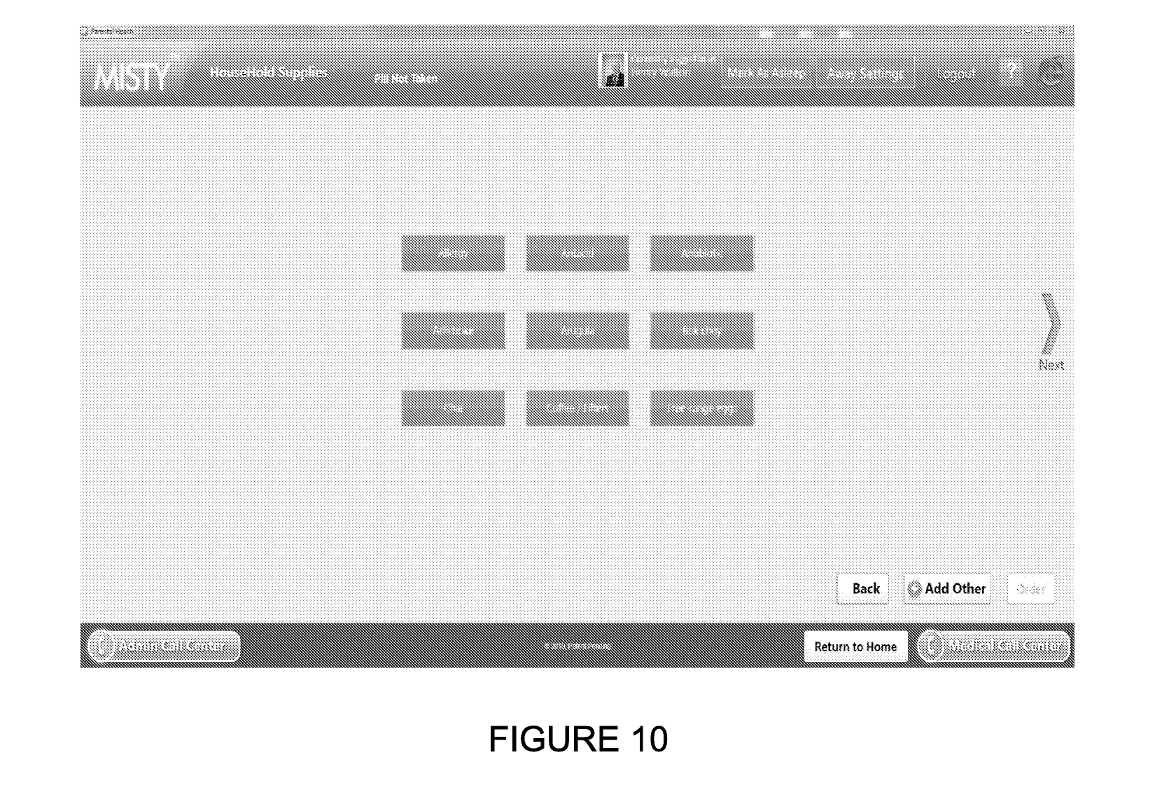

In the exemplary embodiment shown in FIGS. 1-13, the system comprises a touch-screen computing device 100 in the home or residence of the user, whereby the user accesses a series of pages or screens with icons or buttons associated with certain services, tasks, or functions. The computing device also may accept input from other sources, such as a keyboard, mouse, microphone, or the like. Thus, for example, a text message may be typed using a keyboard, voice recognition software, or a virtual keyboard presented on the touchscreen itself. The computing device also may function as a television and video or DVD player. The computing device may be connected to a network and/or the Internet.

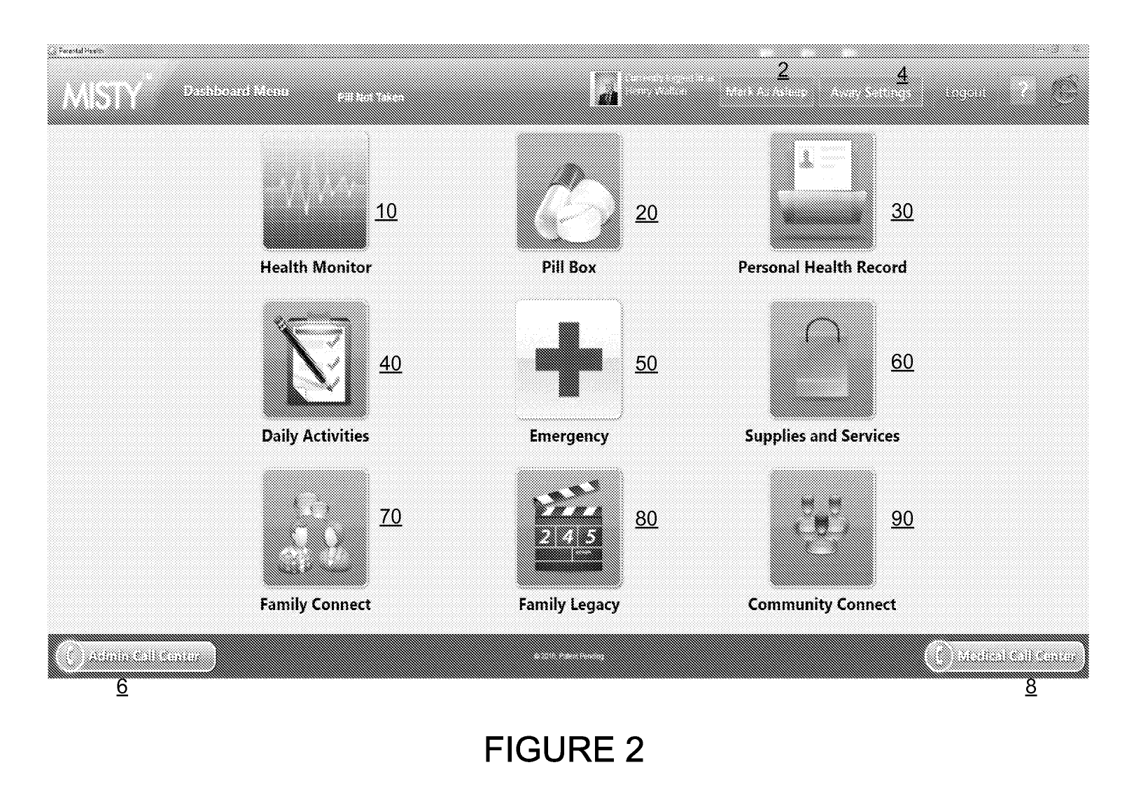

The dashboard menu or “home” screen for an embodiment of the system, as seen in FIGS. 1 and 2, comprises a plurality of primary function buttons or icons 10-90. The user touches the desired button on the touch-screen to implement or launch a desired service or function. All buttons are designed to be easily recognizable by the user, and visible to a senior, and are indicative of the service being launched. Other areas of the touch-screen allow the user to perform other functions (such as returning to the home screen, scanning through multiple screens, scrolling through a list, accessing computing device functions, and the like).

In the embodiment shown in FIG. 2, the user can use a button to indicate that he is asleep 2, change the settings of the system to their “away” status (i.e., when the user is away from home) 4, call the administrative call center 6, or call the medical call center 8.

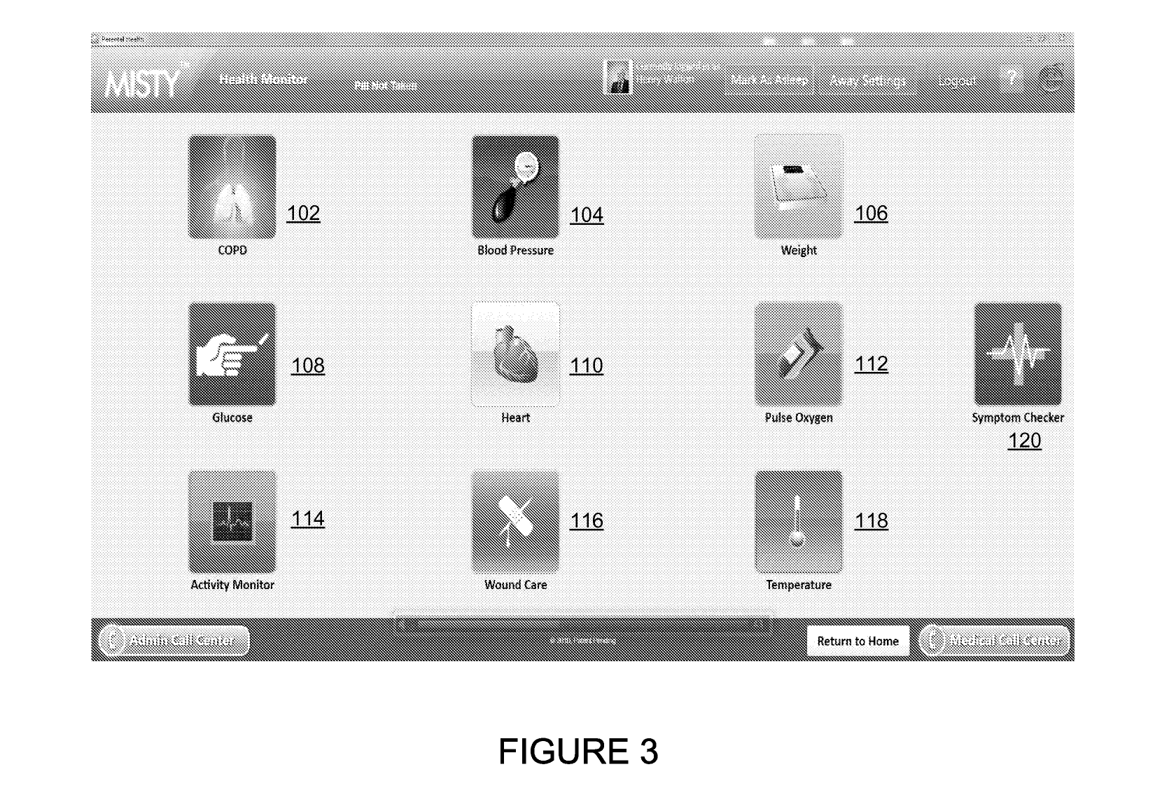

In this particular embodiment, the Health Monitor button 10 leads to a screen (shown in FIG. 3) providing access to routine health monitoring services and reporting. Routine health monitoring services may include, but are not limited to, COPD (chronic obstructive pulmonary disease) 102, blood pressure 104, weight 106, glucose (for diabetic patients) 108, heart monitoring 110, pulse oxygen 112, activity monitor 114, wound care 116, temperature 118, symptom monitoring 120, and other routine health monitoring needs. The system allows the user to perform the necessary monitoring or take measures using standard or off-the-shelf home medical devices 122 (such as a blood glucose monitor or blood pressure cuff). These devices communicate the data to the computing device. Communications may be done wirelessly, or via a wired connection or port (e.g., USB or the like). Some device may have a suitable connector integrated into the device itself (such as a USB connector), and the device may thus be plugged directly into the appropriate port in the computing device.

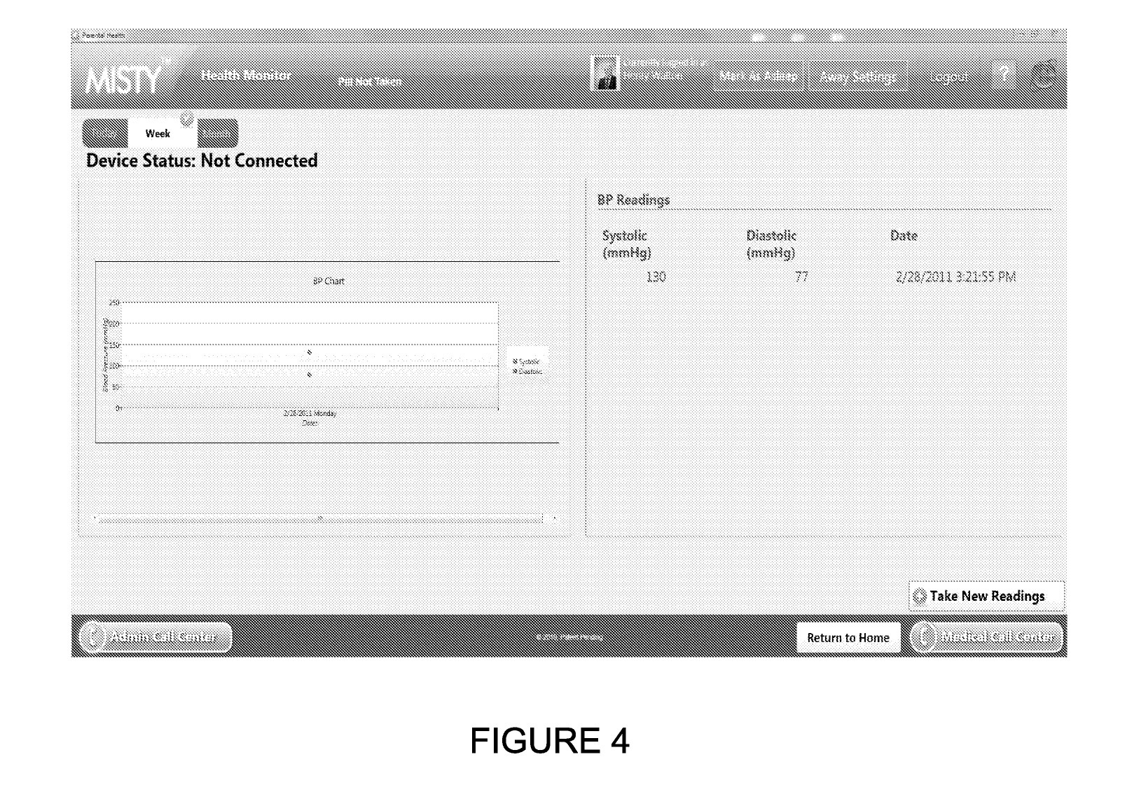

The data is read by the system, and converted to a standard, easily-presented form suitable for the user. An example of a blood pressure data screen is shown in FIG. 4. When the data indicates the user is outside of an established limit or published standards for a particular health parameter, an alert is triggered. The user is then contacted by a nurse or health professional in the remote call center monitoring the system, who triages the patient or user and recommends the next steps for care. The user may communicate with the nurse or health professional over the phone, or using the computing device, or a combination thereof. The computing device may comprise a camera or webcam that can be used to send pictures or video to the nurse or healthcare professional.

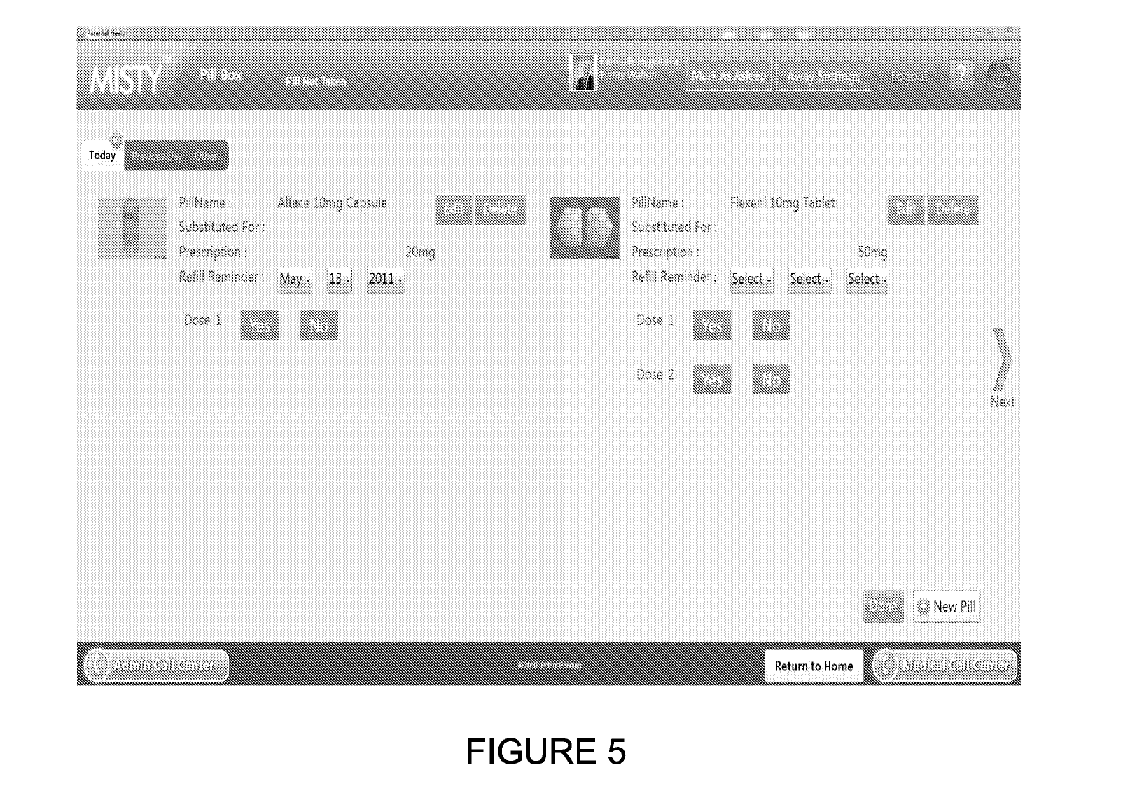

The Pill Box function 20 leads to the Pill Box screen shown in FIG. 5, which reminds the user to take his or her medicine, and prompts the user to explain why it was not taken if the user says the medicine was not taken. In one embodiment, this is a three-step alert system that escalates. The first alert is a visual alarm (such as a flashing message on the touch-screen). The second alert is an audible alarm. The user may directly respond to each of these alerts. The third alert level provides an alert to the remote call center, which prompts a call to the user from the call center. Alternatively, the third alert level may provide notification to the user's family, possibly through a web portal. This function promotes medication compliance and chronic condition management. In other embodiments, this function includes medical drug therapy.

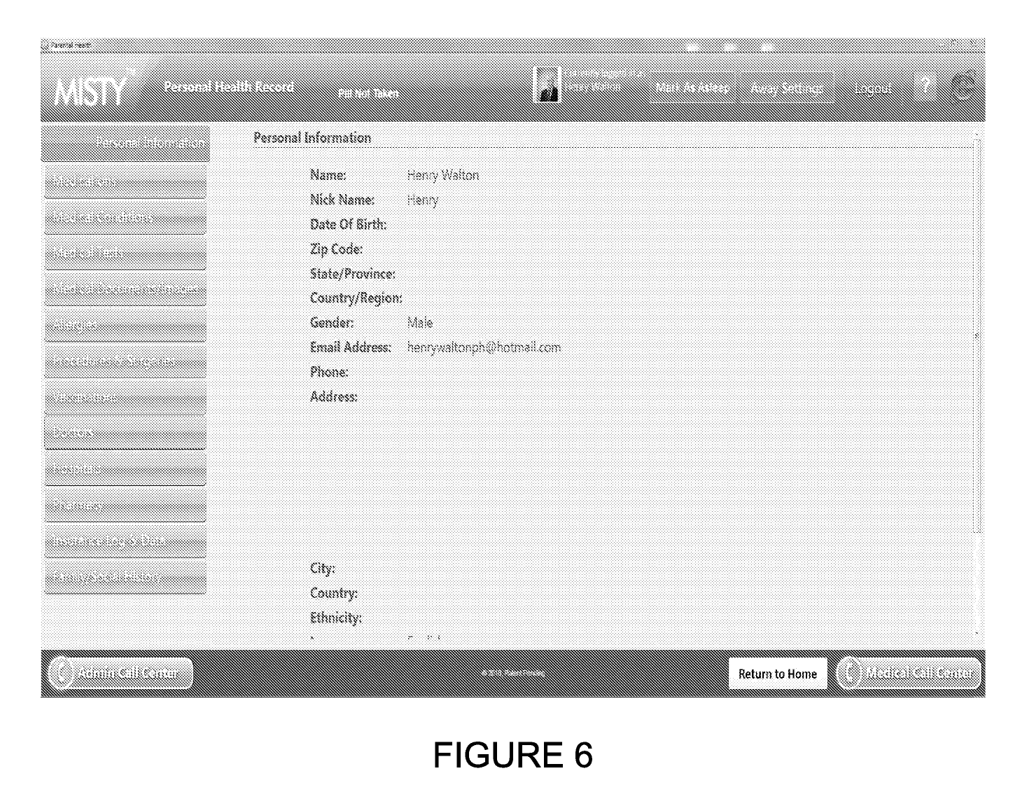

The Personal Health Record function 30 provides the user access to an easy-to-access data management and presentation system (a sample input screen is shown in FIG. 6) that parses the data into simple, easy-to-read forms that the user, or his or her caregiver, can manage and/or retrieve quickly. In one exemplary embodiment, this function provides an interface to the Microsoft Health Vault and other systems.

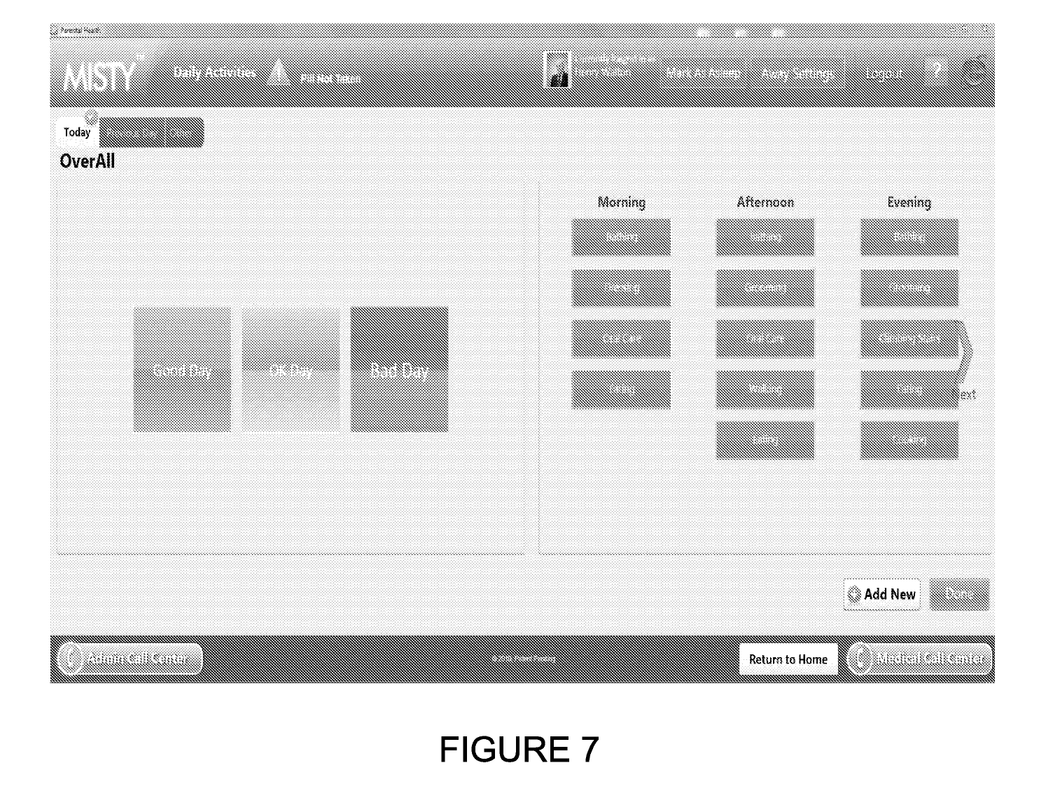

The Daily Activities function 40 allows the user to update his or her family (and possibly friends) on daily activities or current status via a family webpage or network, Twitter, text message, Facebook, email, or other similar communication means. In one variation, as seen in FIG. 7, the user can simply touch a button or buttons on the screen to indicate a general status or particular activity (e.g., “Good Day”, “Bad Day”, “OK Day”, “Nap”, “Exercise”, “Meal”, etc.).

The Emergency function 50 allows the user to dial 911 (or other emergency numbers), but with the added functionality of having a webcam and also access to the Personal Health Record, Pill Box, Health Monitoring and Daily Activities information. For example, a nurse or health professional in the call center has the ability to provide key information to the 911 operator, EMT, or Emergency Room, with the patient's permission.

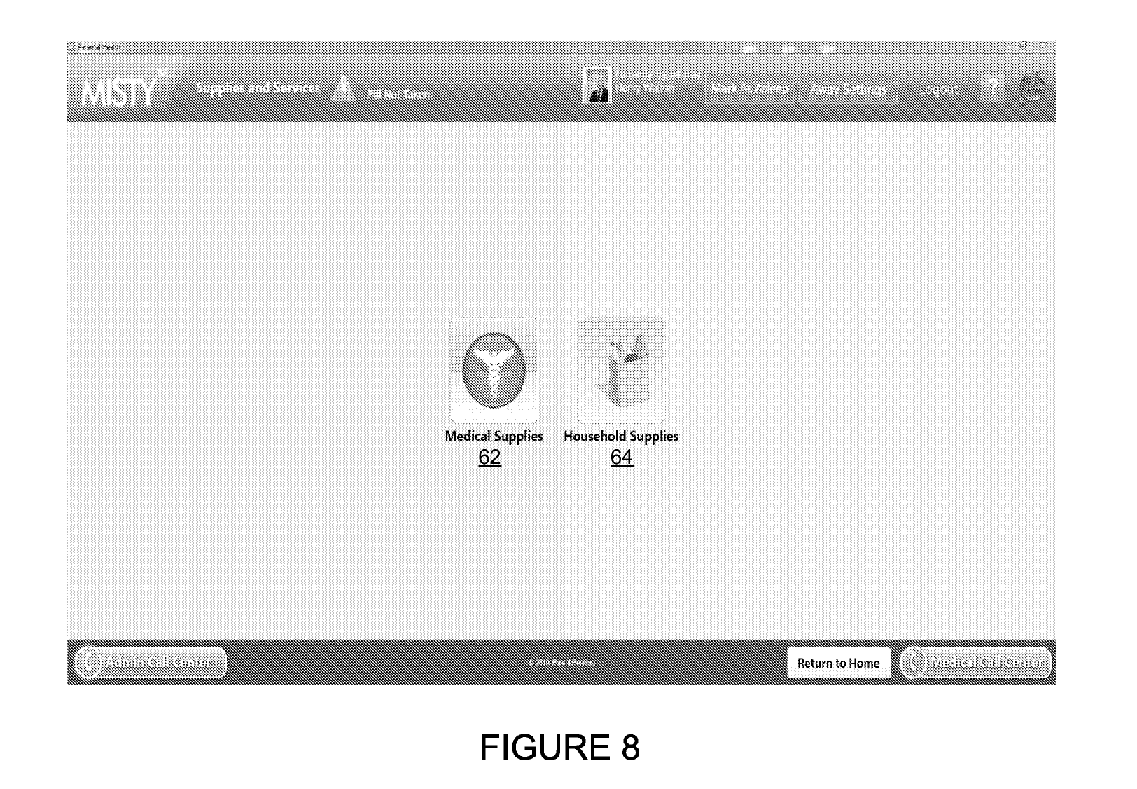

The Supplies, or Supplies and Services, function 60 gives the user the ability to order medical supplies 62, groceries, or other household supplies 64 via an EDI (Electronic Data Interchange) interface with suppliers and vendors, as seen in FIGS. 8-10.

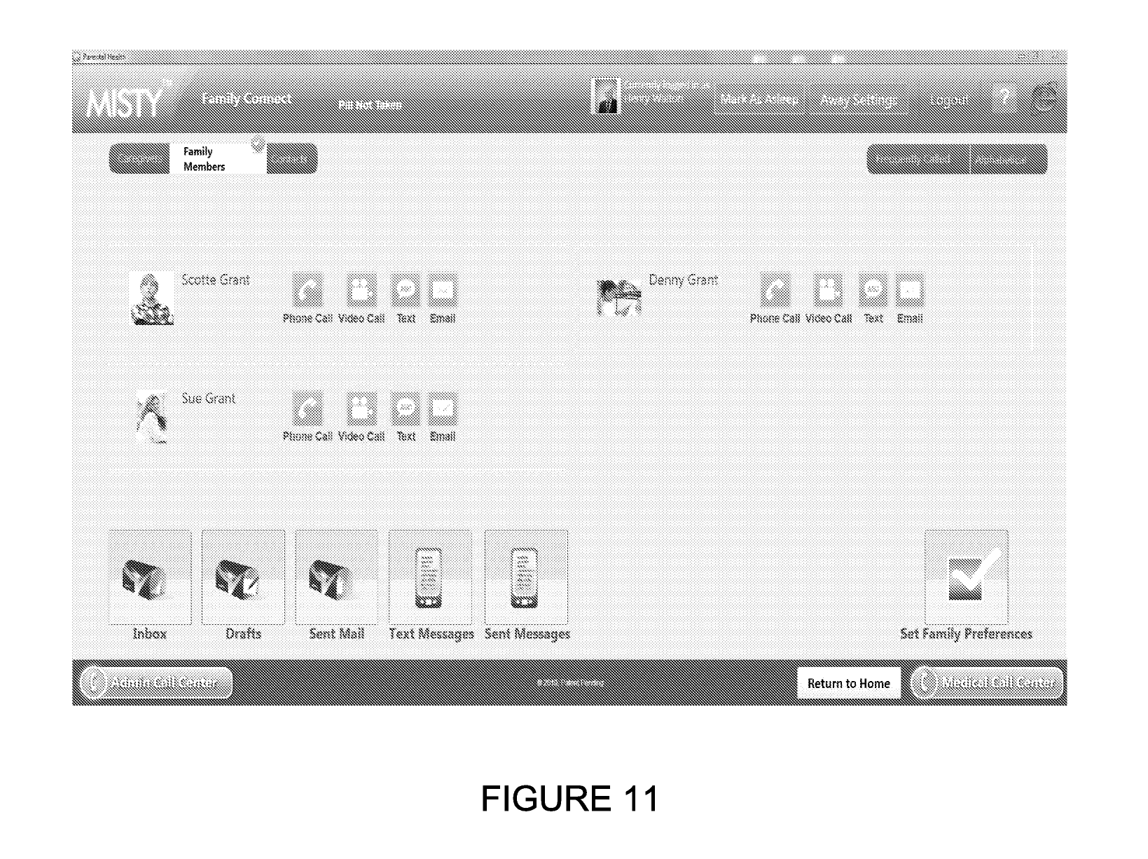

The Family Connect function 70 provides easy one-touch calling to or communications with family or friends. FIG. 11 shows an exemplary embodiment of a Family Connect screen. The call may be a video call using the webcam. This function also may provide the user the ability to text or email family members by using the touch screen, a keyboard, or voice recognition software.

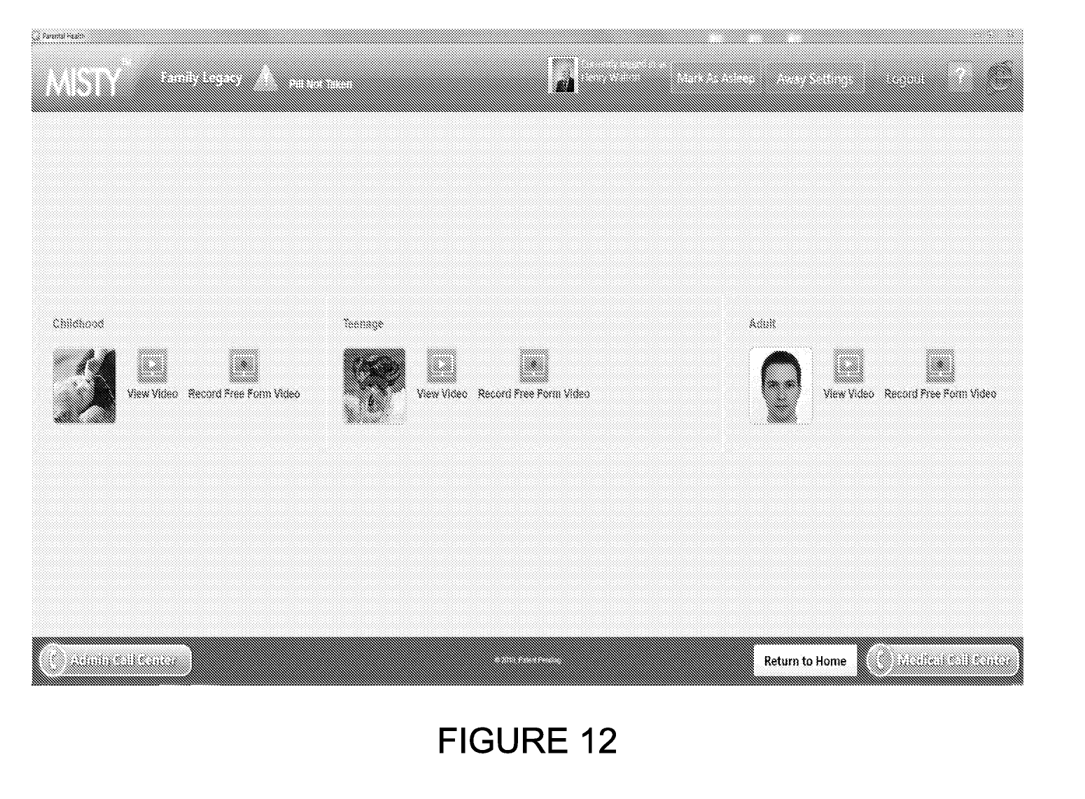

The Family Legacy function 80 allows the user to record, store, and publish or share videos or pictures recorded by the webcam, as seen in FIG. 12. By this means, a user can record a video or sound recording of his or her memories or stories. In one embodiment, the system provides a series of questions to guide the response or recording.

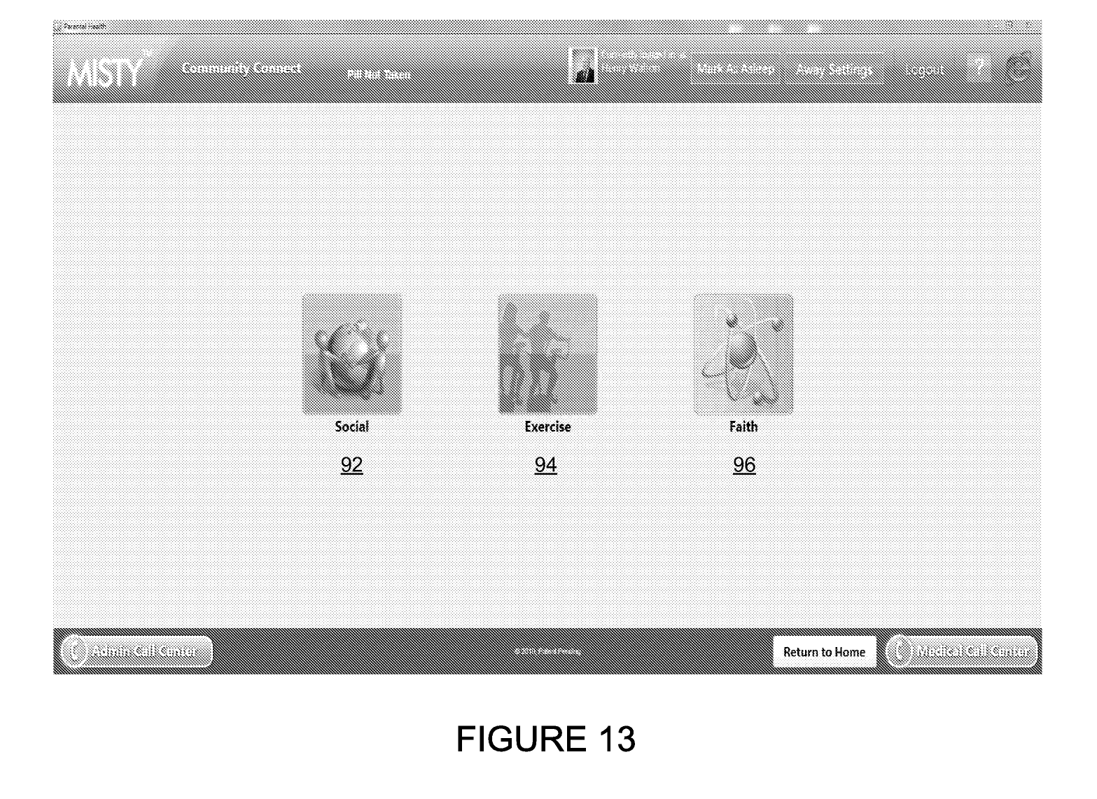

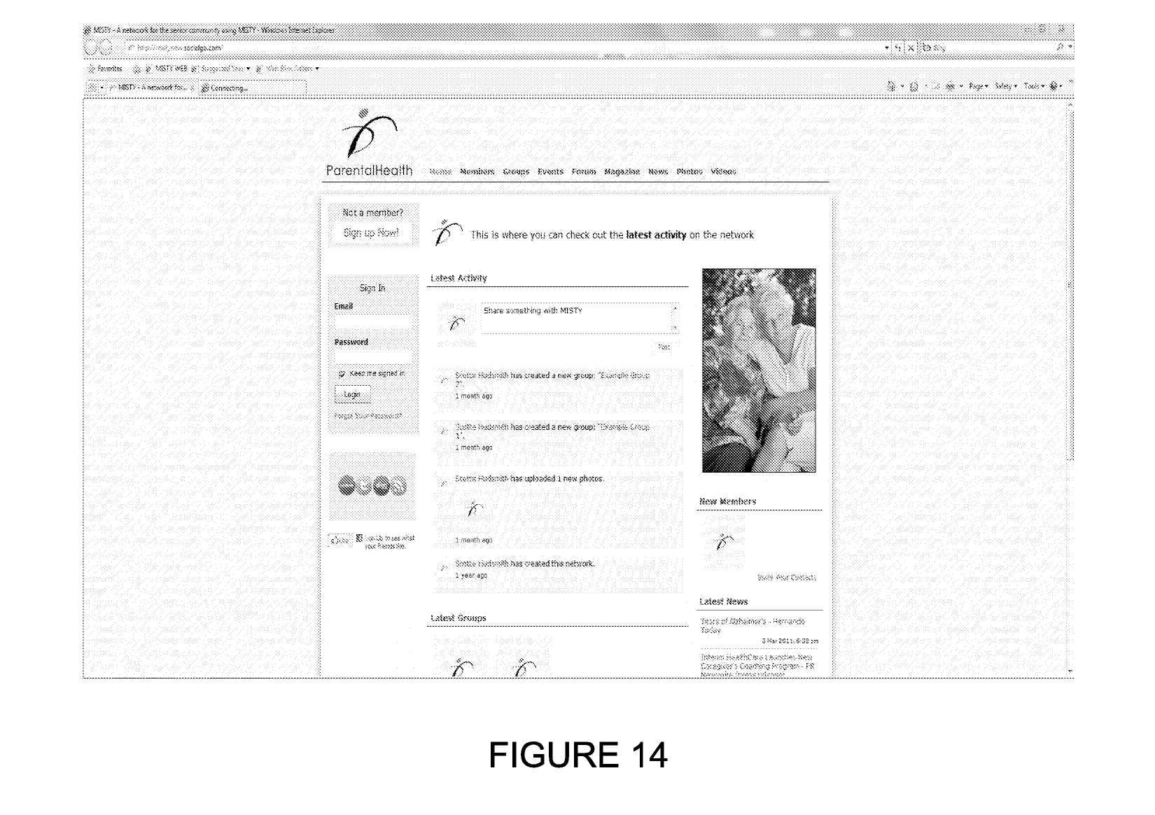

The Community Connect function 90 provides several options for a user to connect to the community, as seen in FIG. 13. One exemplary function is a social networking feature 92, similar to Facebook (as seen in FIG. 14). Another connects a senior user to other seniors. Another exemplary function connects the user to a community of their faith 96. And yet another connects the user to an exercise or health network 94 to promote and improve well-being.

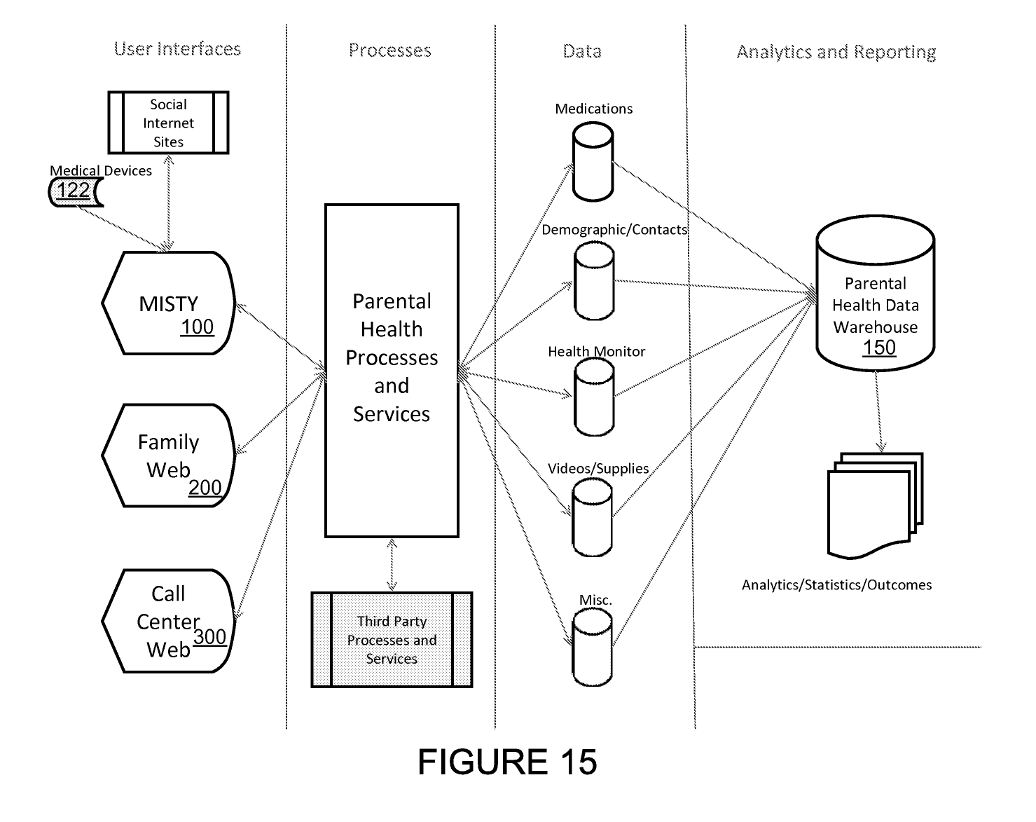

FIG. 15 shows another embodiment of a system in accordance with the present invention, comprising the above-described touch-screen computing device user interface 100, which is networked communication with a family web application 200 and a call center web application 300. These applications provide processes and services to the users, their families, and system administrators and managers. Data is stored in one or more databases or data warehouses 150.

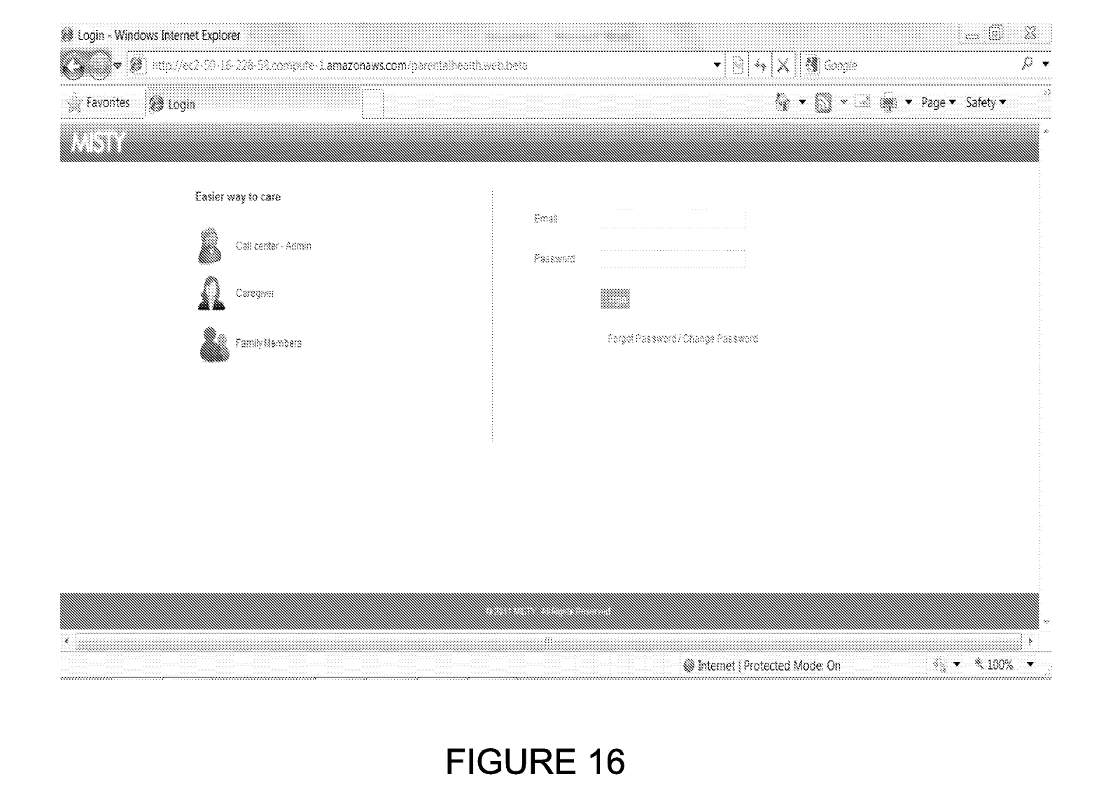

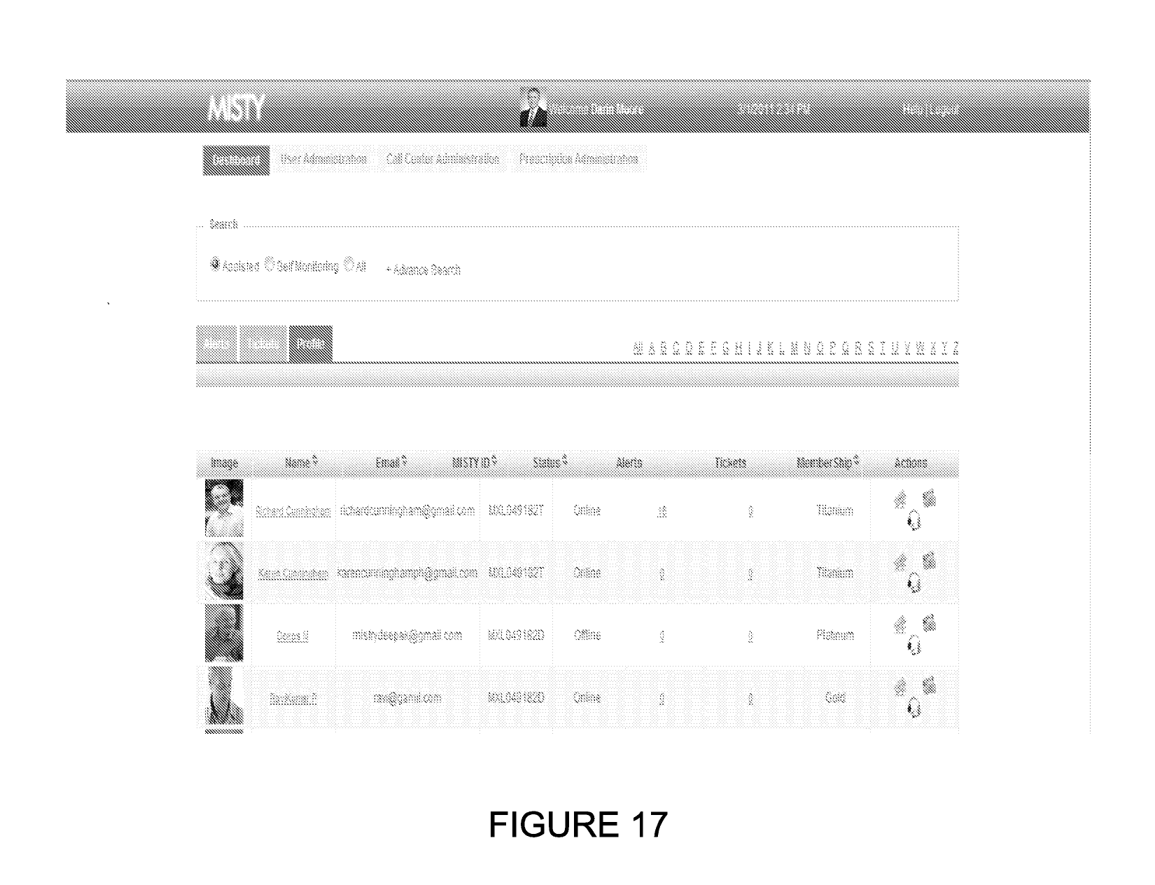

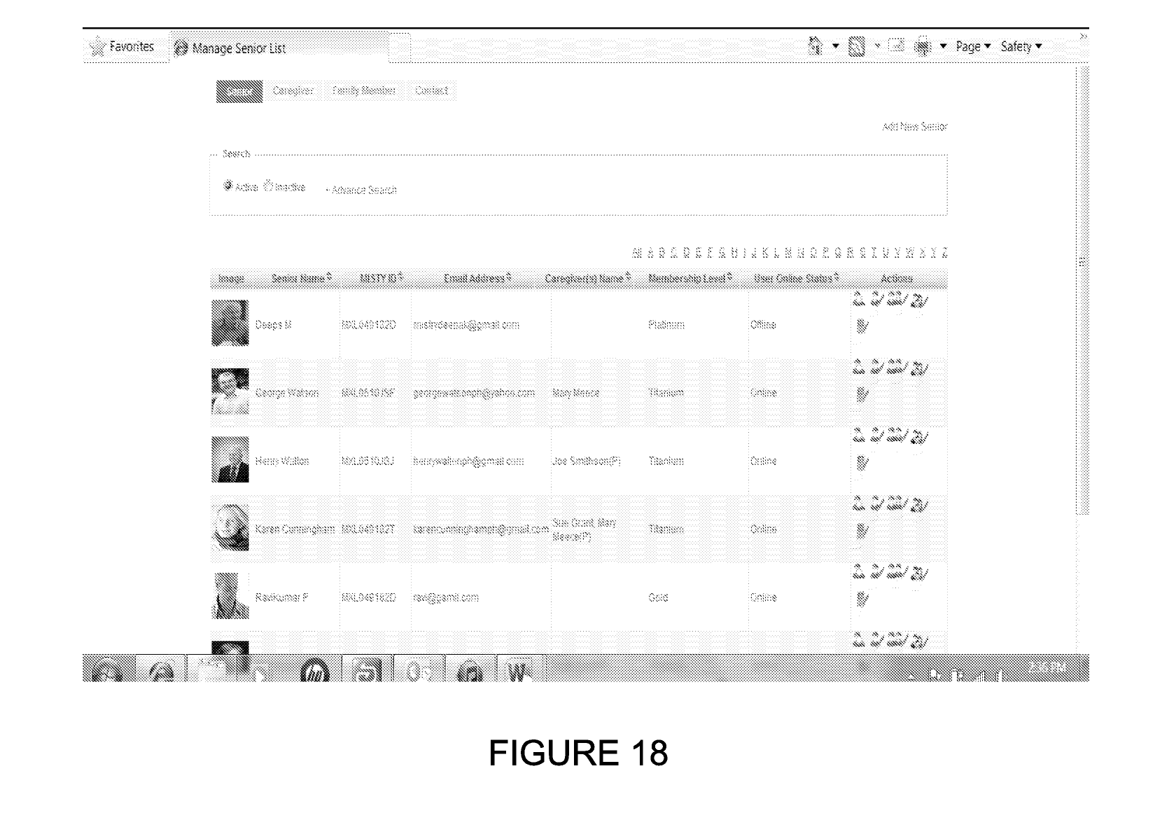

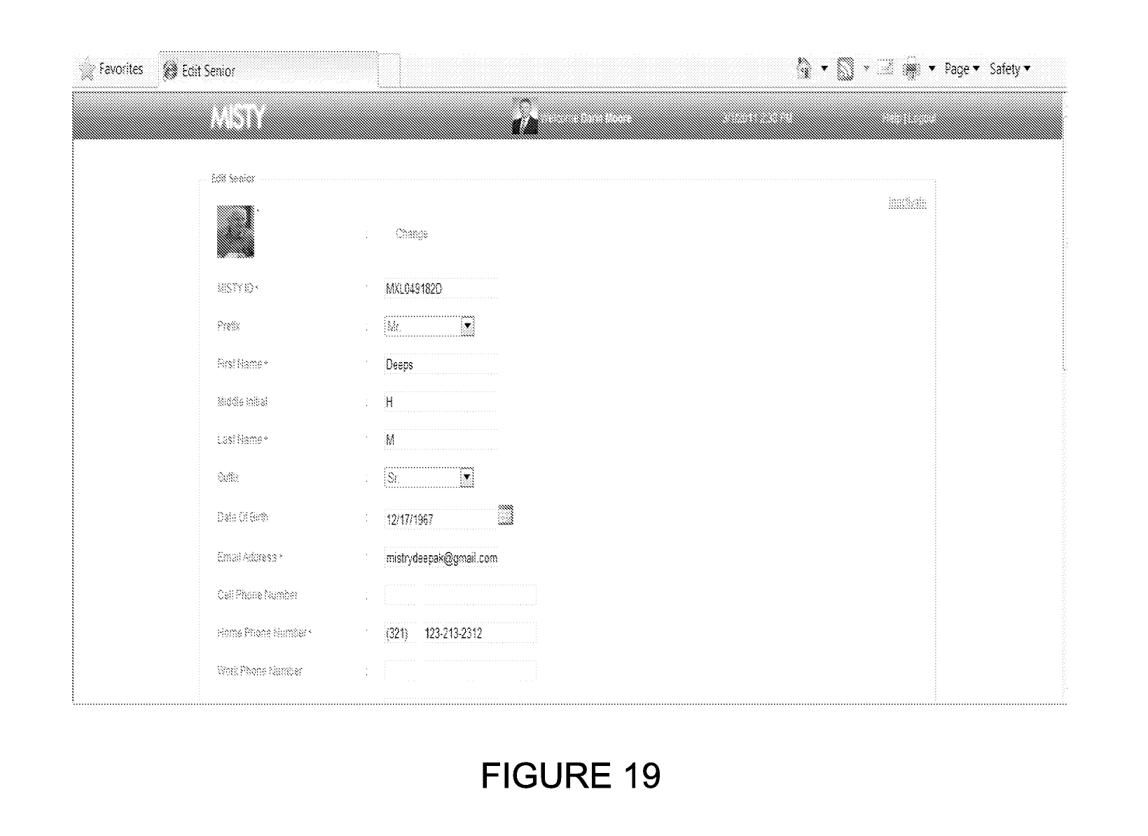

FIGS. 16 through 24 shows exemplary embodiments of screens from the call center web application 200, used to administer or manage the system. FIG. 16 shows a log in screen, while FIG. 17 shows a general user dashboard with specific individual users. Information available in the dashboard include a picture, name, email, identification number, status, number of alerts, number of tickets, level of membership in the system, and current actions. Individuals can be sorted by those doing self-monitoring and those being assisted by others. FIG. 18 shows a list of senior users, although the administrative user also can view caregivers and family members. FIG. 19 shows a screen for adding or editing information about a senior user.

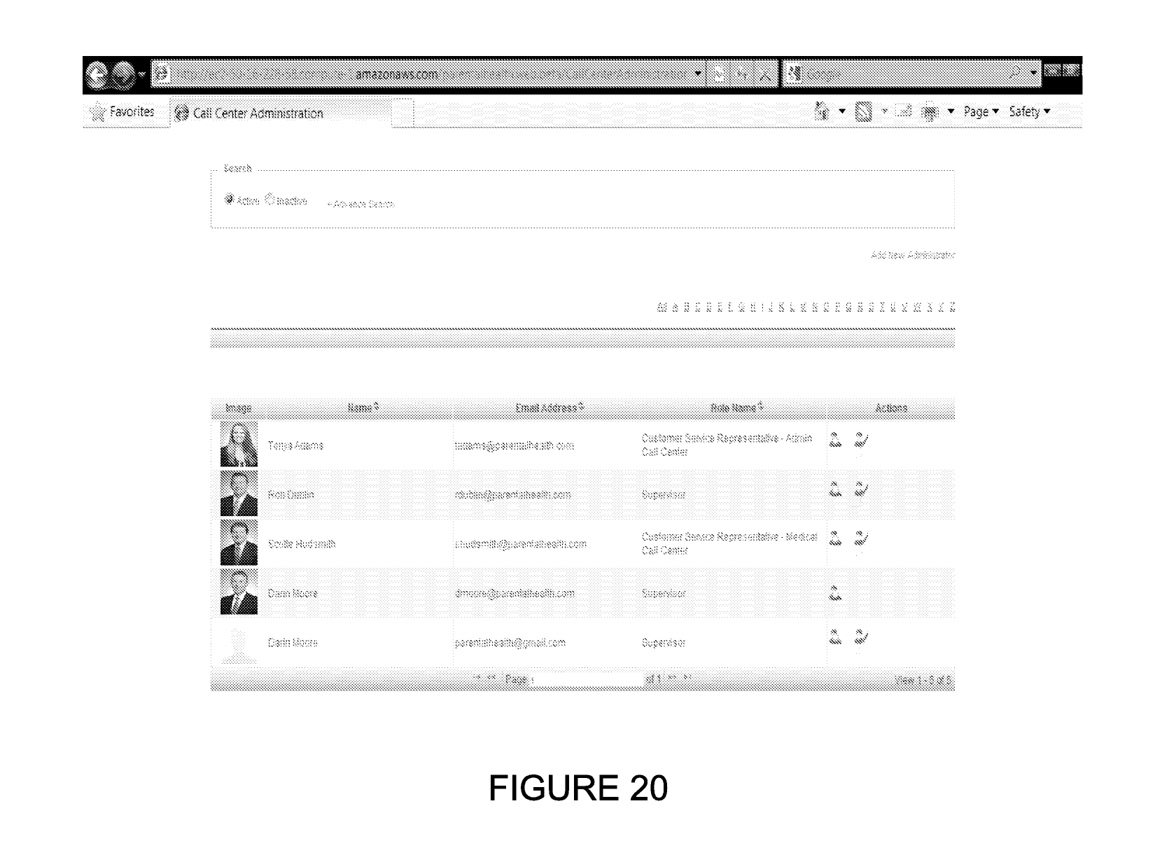

FIG. 20 shows a list of call center administrative or system-level users. Information presented may include names, pictures, emails, role names, and current actions.

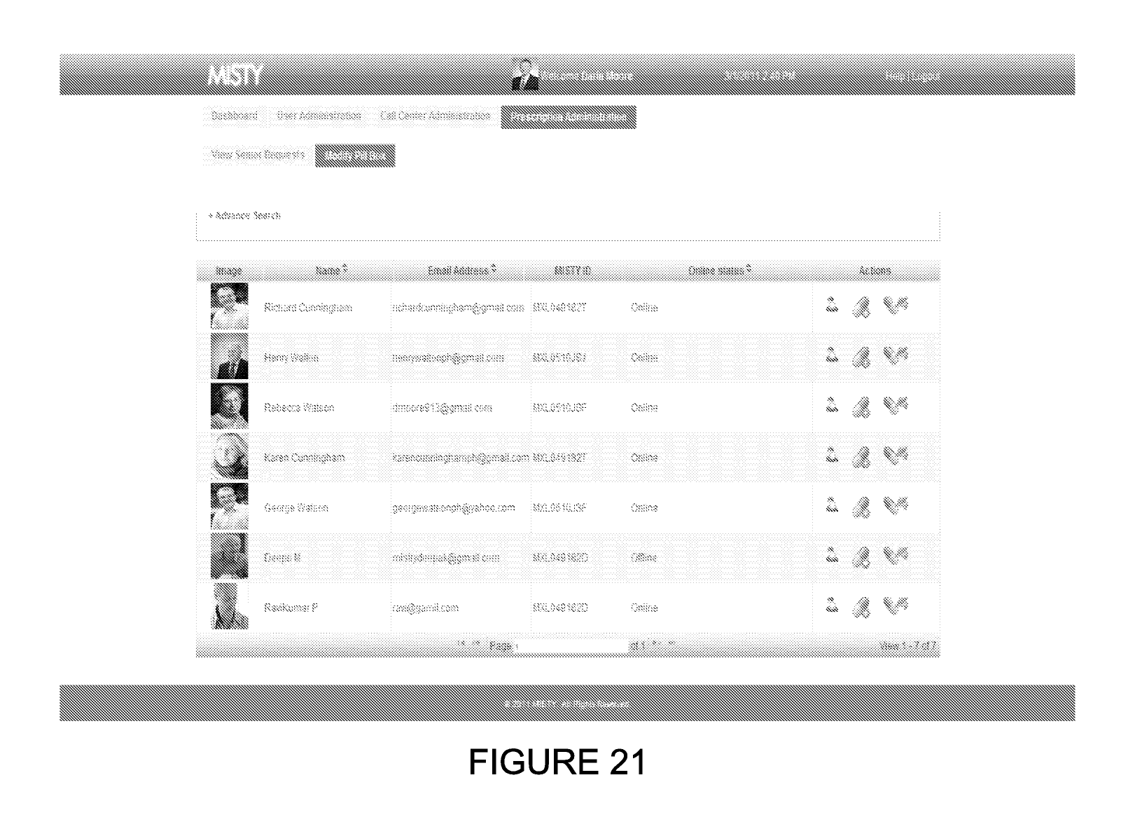

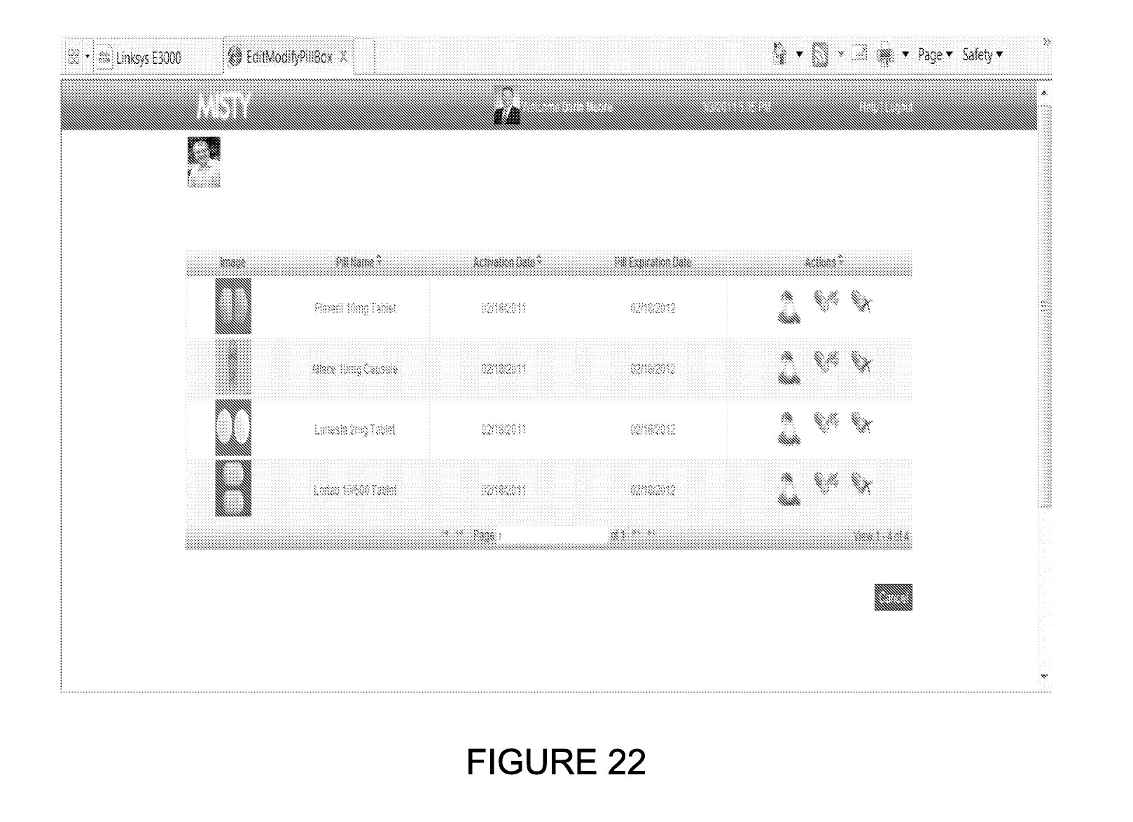

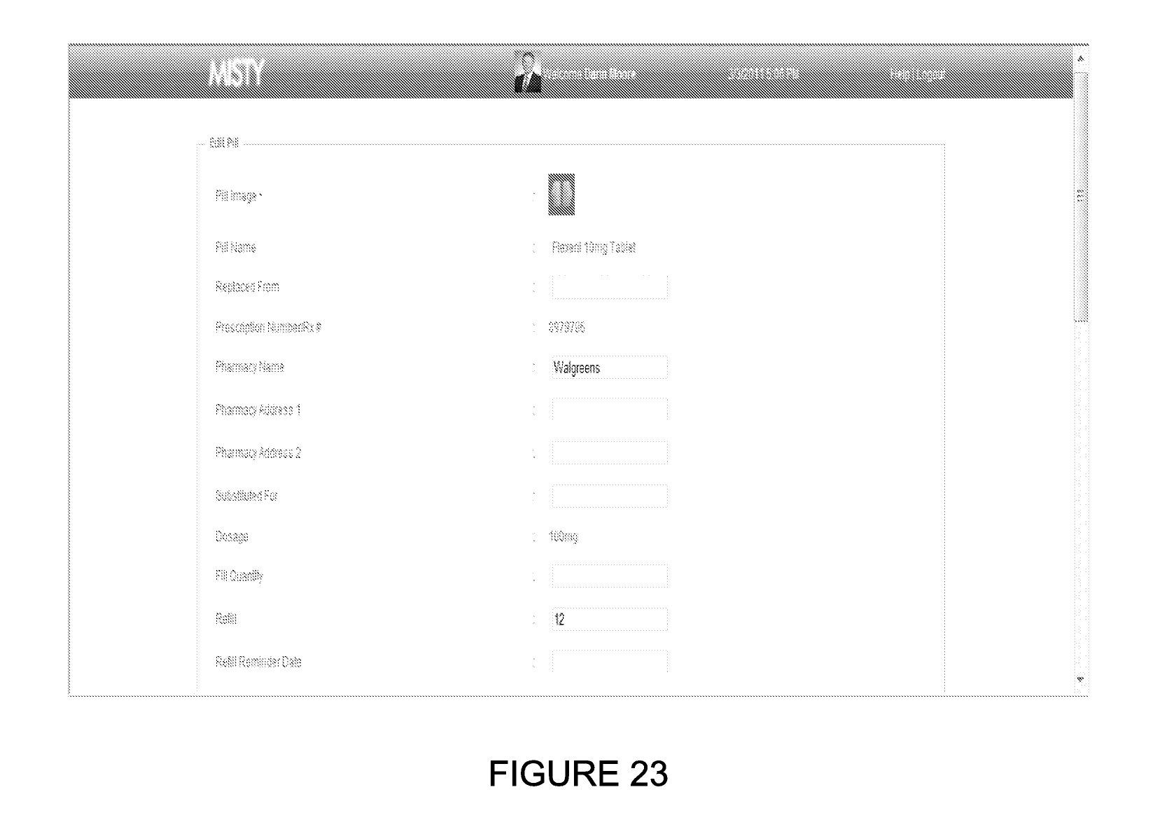

FIG. 21 shows a screen for prescription (or “pill box”) administration. FIG. 22 shows a “pill box modification” screen for an individual, while FIG. 23 shows details on any particular type of pill or pills.

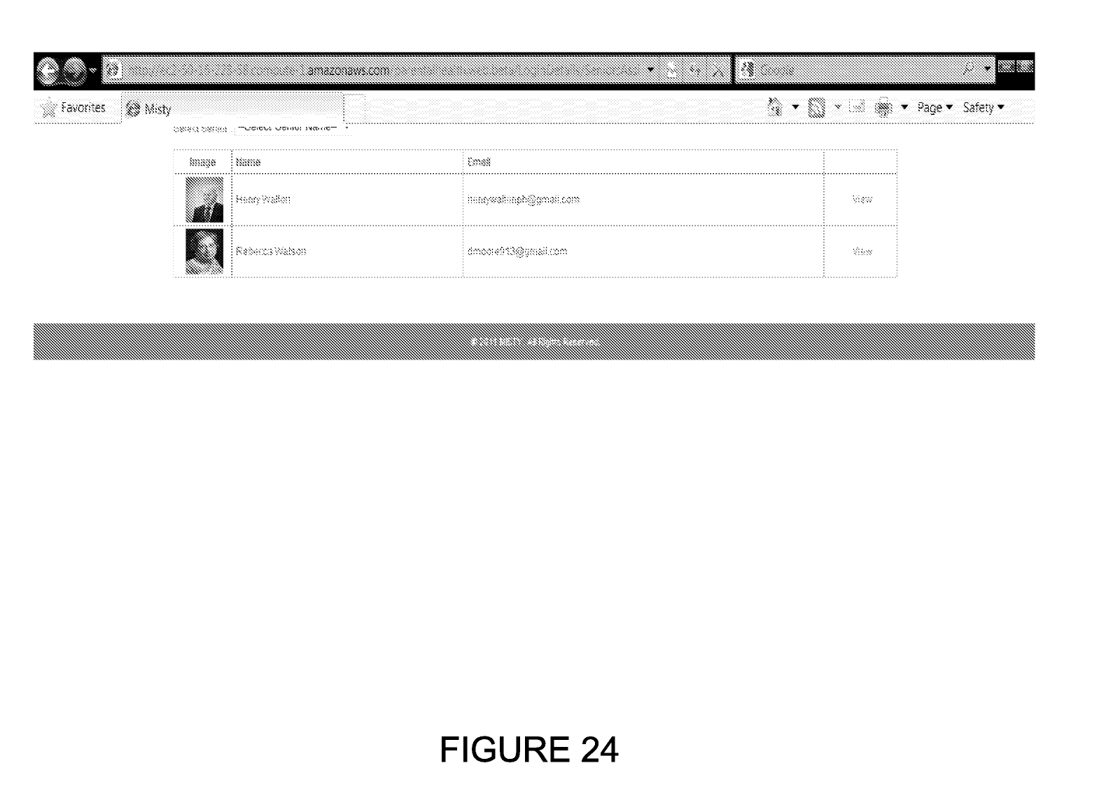

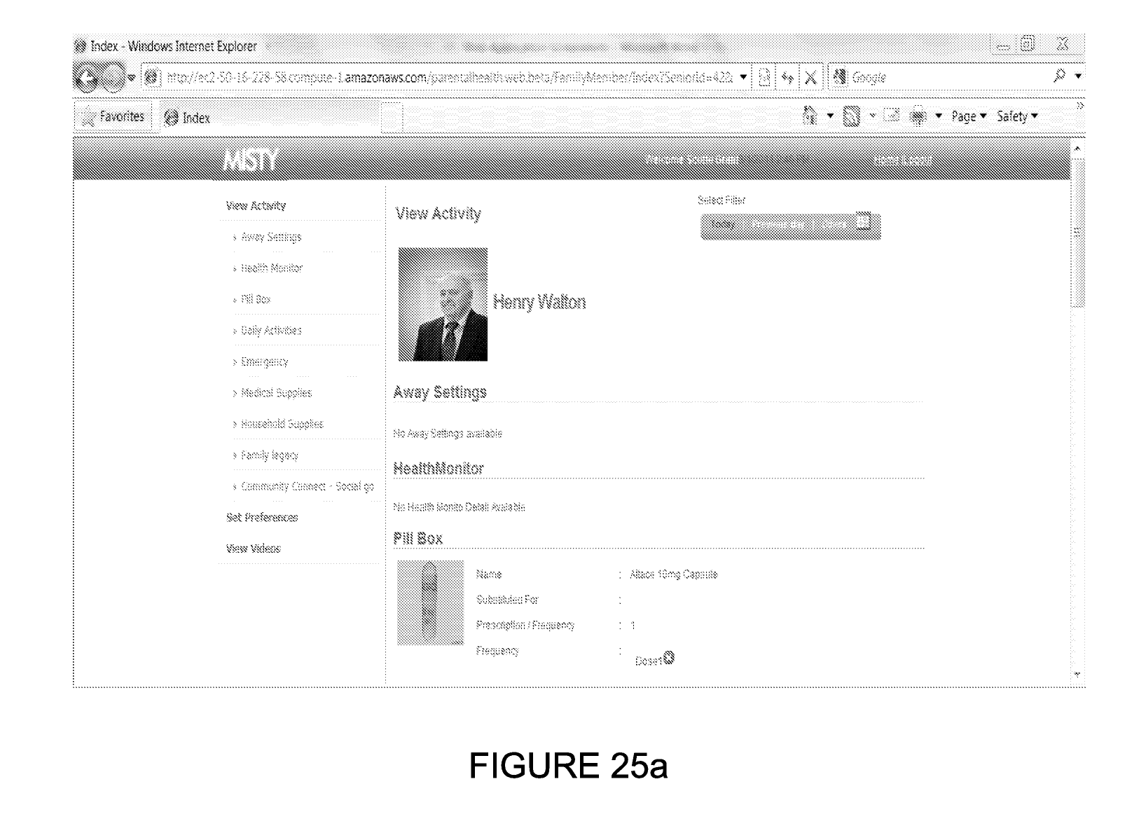

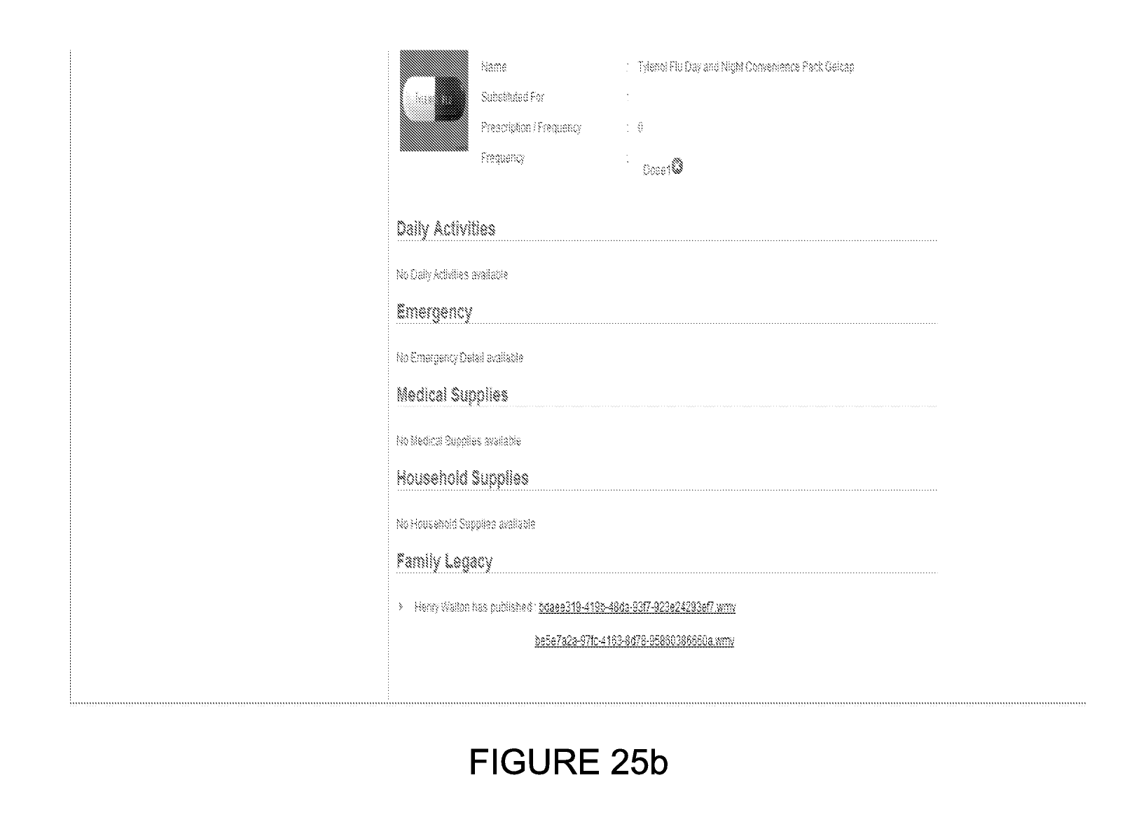

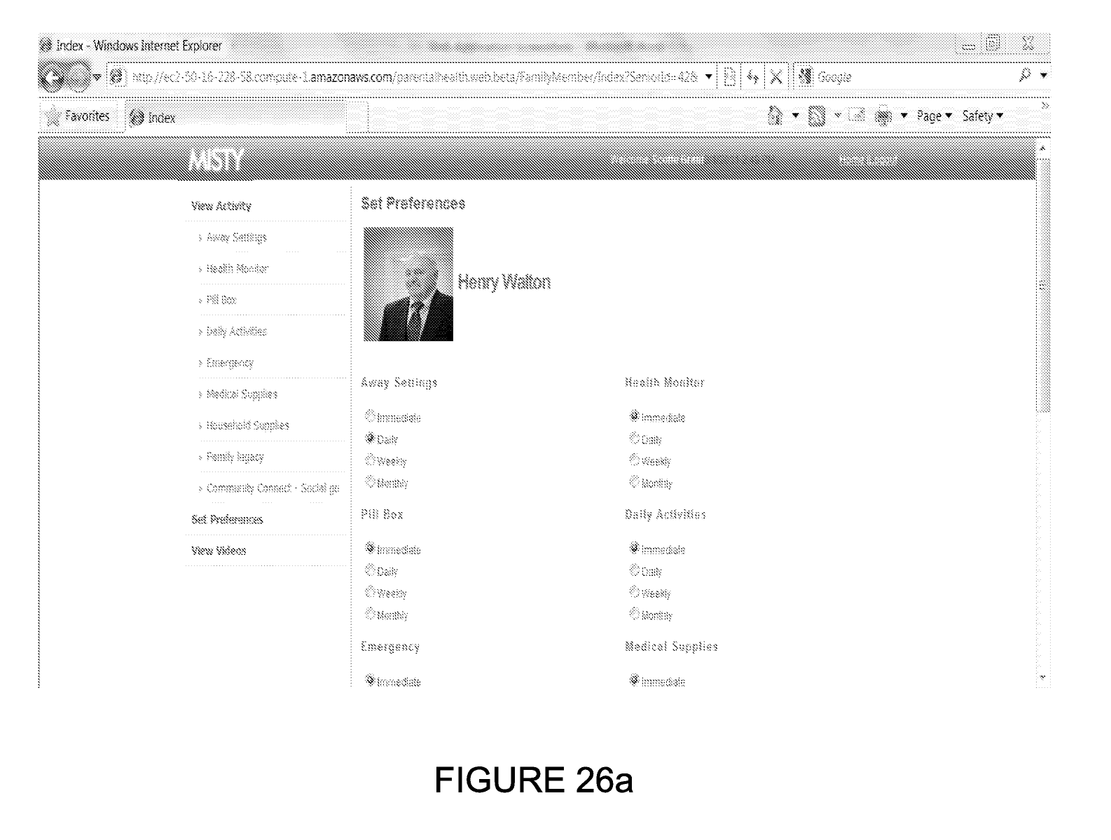

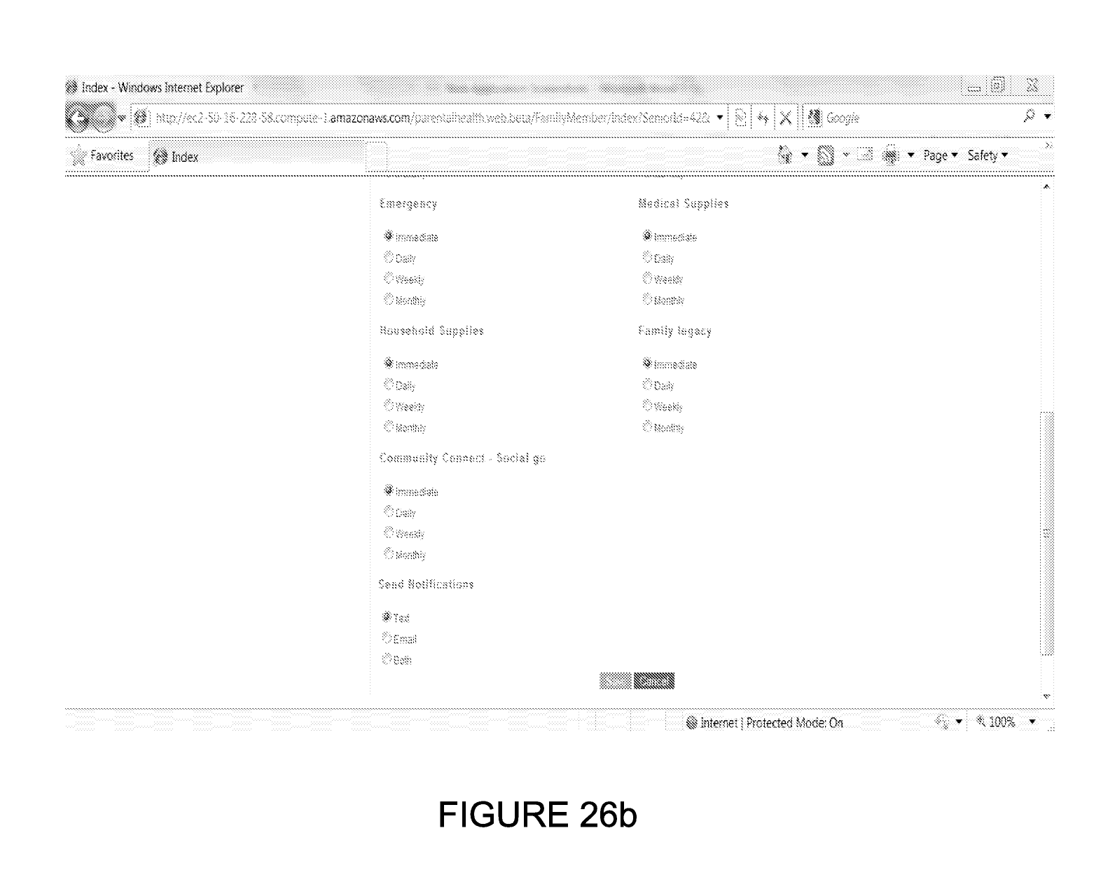

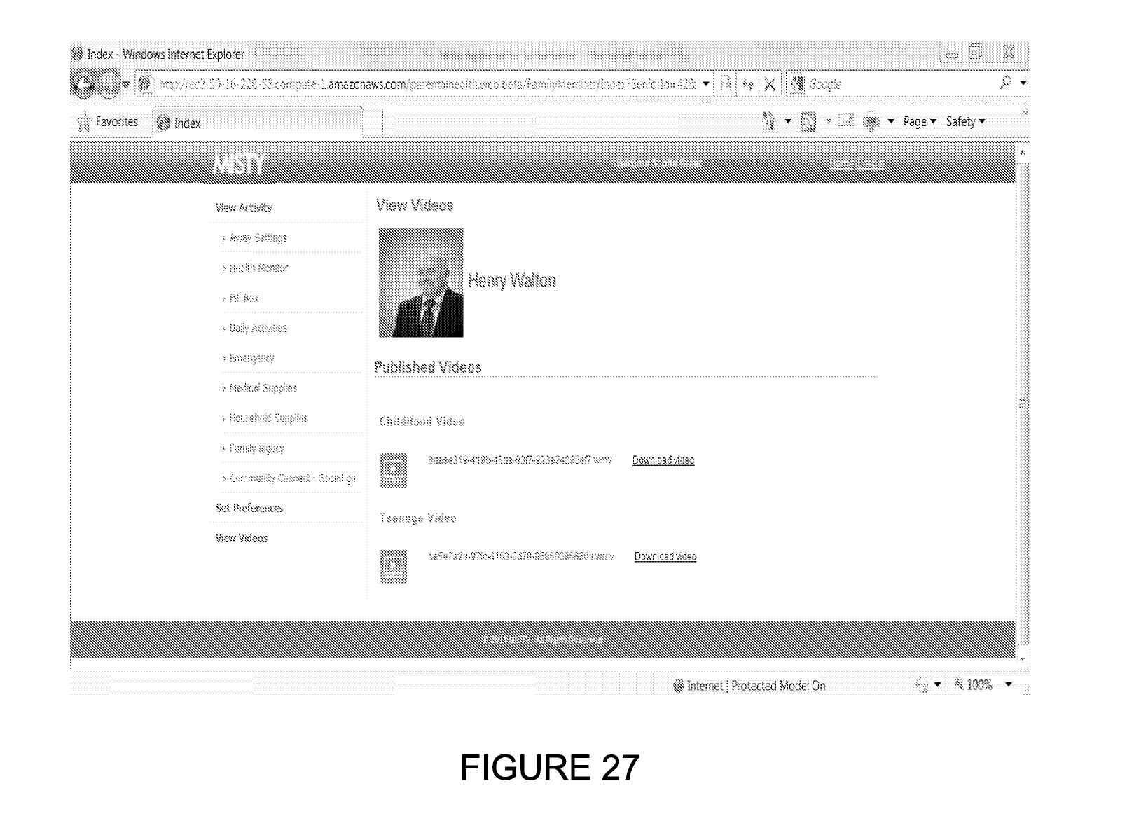

FIGS. 24 through 27 show exemplary screens from an embodiment of the family web application 300. This is the user interface that family members can use to check on and communicate with a senior parent or other user of the system. FIG. 24 provides a list of those users a particular family member can access information about. FIGS. 25a-b provide an information summary for the selected user (in one embodiment, this is the same screen, and a scroll bar can be used to move from top to bottom). FIGS. 26a-b allows the family member user to set preferences for the selected user. FIG. 27 shows a screen for a family member user to view a video or hear a recording from the selected individual.

Accordingly, the present invention provides a system to support seniors who desire to “age in place,” disabled individuals, or other users, thereby improving the quality of life for people who are capable of living independently in their homes, residences, senior living facilities, or elsewhere, but who also need regular health monitoring. A user can, for example, check vitals (e.g., temperature, blood pressure) every morning, and make sure the day's medications are administered. They can monitor indicators for common diseases such as diabetes and heart problems. They can update an electronic health record and send it to their physician. They can order medical supplies that are delivered directly to the home. They are connected with a 24-hour, 7 days a week support network of nurses and health professionals with the clinical expertise to handle everything from an upset stomach to a life-threatening emergency. They also can keep connected with family, friends and faith groups, report details on their health and daily activities, send emails and text messages to grandchildren and family members, and record video messages to post on a secure web site.

The system is simple, with one primary self-contained device that requires only an Internet connection. The device is large, easy to see, and user-friendly, with a built-in web camera connecting users visually and audibly to clinical call centers. It is reliable, with daily health monitoring driven by alerts and alarms, with direct connections to emergency services when needed. It can be provided as a monthly service for less than the cost of a typical in-home call by a nurse or other healthcare professional. It also has the benefit of providing care without needed readmission of the patient to a healthcare facility (which can cause the facility to incur expenses without being paid or reimbursed for such, or causing the patient or user to have to pay such).

In order to provide a context for the various aspects of the invention, the following discussion provides a brief, general description of a suitable networked computing environment in which the various aspects of the present invention may be implemented. A computing system environment is one example of a suitable computing environment, but is not intended to suggest any limitation as to the scope of use or functionality of the invention. A computing environment may contain any one or combination of components discussed below, and may contain additional components, or some of the illustrated components may be absent. Various embodiments of the invention are operational with numerous general purpose or special purpose computing systems, environments or configurations. Examples of computing systems, environments, or configurations that may be suitable for use with various embodiments of the invention include, but are not limited to, personal computers, laptop computers, computer servers, computer notebooks, tablets, hand-held devices, microprocessor-based systems, multiprocessor systems, TV set-top boxes and devices, programmable consumer electronics, cell phones, personal digital assistants (PDAs), network PCs, minicomputers, mainframe computers, embedded systems, distributed computing environments, and the like.

Embodiments of the invention may be implemented in the form of computer-executable instructions, such as program code or program modules, being executed by a computer or computing device. Program code or modules may include programs, objections, components, data elements and structures, routines, subroutines, functions and the like. These are used to perform or implement particular tasks or functions. Embodiments of the invention also may be implemented in distributed computing environments. In such environments, tasks are performed by remote processing devices linked via a communications network or other data transmission medium, and data and program code or modules may be located in both local and remote computer storage media including memory storage devices.

In one embodiment, a computer system comprises multiple client devices in communication with at least one server device through or over a network. In various embodiments, the network may comprise the Internet, an intranet, Wide Area Network (WAN), or Local Area Network (LAN). It should be noted that many of the methods of the present invention are operable within a single computing device.

A client device may be any type of processor-based platform that is connected to a network and that interacts with one or more application programs. The client devices each comprise a computer-readable medium in the form of volatile and/or nonvolatile memory such as read only memory (ROM) and random access memory (RAM) in communication with a processor. The processor executes computer-executable program instructions stored in memory. Examples of such processors include, but are not limited to, microprocessors, ASICs, and the like.

Client devices may further comprise computer-readable media in communication with the processor, said media storing program code, modules and instructions that, when executed by the processor, cause the processor to execute the program and perform the steps described herein. Computer readable media can be any available media that can be accessed by computer or computing device and includes both volatile and nonvolatile media, and removable and non-removable media. Computer-readable media may further comprise computer storage media and communication media. Computer storage media comprises media for storage of information, such as computer readable instructions, data, data structures, or program code or modules. Examples of computer-readable media include, but are not limited to, any electronic, optical, magnetic, or other storage or transmission device, a floppy disk, hard disk drive, CD-ROM, DVD, magnetic disk, memory chip, ROM, RAM, EEPROM, flash memory or other memory technology, an ASIC, a configured processor, CDROM, DVD or other optical disk storage, magnetic cassettes, magnetic tape, magnetic disk storage or other magnetic storage devices, or any other medium from which a computer processor can read instructions or that can store desired information. Communication media comprises media that may transmit or carry instructions to a computer, including, but not limited to, a router, private or public network, wired network, direct wired connection, wireless network, other wireless media (such as acoustic, RF, infrared, or the like) or other transmission device or channel. This may include computer readable instructions, data structures, program modules or other data in a modulated data signal such as a carrier wave or other transport mechanism. Said transmission may be wired, wireless, or both. Combinations of any of the above should also be included within the scope of computer readable media. The instructions may comprise code from any computer-programming language, including, for example, C, C++, C#, Visual Basic, Java, and the like.

Components of a general purpose client or computing device may further include a system bus that connects various system components, including the memory and processor. A system bus may be any of several types of bus structures, including, but not limited to, a memory bus or memory controller, a peripheral bus, and a local bus using any of a variety of bus architectures. Such architectures include, but are not limited to, Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computing and client devices also may include a basic input/output system (BIOS), which contains the basic routines that help to transfer information between elements within a computer, such as during start-up. BIOS typically is stored in ROM. In contrast, RAM typically contains data or program code or modules that are accessible to or presently being operated on by processor, such as, but not limited to, the operating system, application program, and data.

Client devices also may comprise a variety of other internal or external components, such as a monitor or display, a keyboard, a mouse, a trackball, a pointing device, touch pad, microphone, joystick, satellite dish, scanner, a disk drive, a CD-ROM or DVD drive, medical devices, or other input or output devices. These and other devices are typically connected to the processor through a user input interface coupled to the system bus, but may be connected by other interface and bus structures, such as a parallel port, serial port, game port or a universal serial bus (USB). A monitor or other type of display device is typically connected to the system bus via a video interface. In addition to the monitor, client devices may also include other peripheral output devices such as speakers and printer, which may be connected through an output peripheral interface.

Client devices may operate on any operating system capable of supporting an application of the type disclosed herein. Client devices also may support a browser or browser-enabled application. Examples of client devices include, but are not limited to, personal computers, laptop computers, personal digital assistants, computer notebooks, hand-held devices, cellular phones, mobile phones, smart phones, pagers, digital tablets, Internet appliances, and other processor-based devices. Users may communicate with each other, and with other systems, networks, and devices, over the network through the respective client devices.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A system for health and activity monitoring, comprising:
a computing device with a computer processor or microprocessor, wherein the microprocessor is programmed to present on a display a user with a plurality of services or options, further wherein the plurality of services or options comprises the following:
a health monitoring service, which when selected presents the user with a health monitoring option screen with a plurality of specific health care functions; and
a medication option, which when selected assists the user in taking and managing their medication.

2. The system of claim 1, wherein the specific health care functions include one or more of the following options: chronic obstructive pulmonary disease; blood pressure; weight; glucose; heart monitoring; pulse oxygen; activity monitor; wound care; temperature; and symptom monitoring.

3. The system of claim 2, wherein the computing device receives information from a medical device used by the user for a specific health care function.

4. The system of claim 3, wherein the information is received via wired communication or wireless communication.

5. The system of claim 3, wherein the information received is presented on the display.

6. The system of claim 3, wherein the computing device triggers an alert if the information received is outside of an established limit or published standard for an applicable health parameter, and initiates contact with a healthcare professional or monitor in a remote location.

7. The system of claim 1, further comprising a camera or webcam.

8. The system of claim 1, wherein the computing device comprises a touchscreen display.

9. The system of claim 1, wherein the medication option presents a screen that reminds the user to take his or her medicine.

10. The system of claim 9, wherein the computing device presents a series of alarms if the user does not respond to the system's reminders to take medicine, or indicates that the medicine was not taken but does not provide an appropriate explanation.

11. The system of claim 10, wherein the computing device provides an alert to a healthcare professional or monitor in a remote location if the medication is not taken.

12. The system of claim 10, wherein the computing device provides an alert to a family member of the user if the medication is not taken.

13. The system of claim 1, wherein the plurality of services or options further comprises one or more of the following:
a personal health record option, which when selected prompts the user to enter or edit personal health record information;
a daily activities option, which when selected allows the user to indicate his or her general status or particular activities the user is performing;
an emergency function, which when selected allows the user to contact emergency services;
a supplies and services function, which when selected allows the user to order medical, food, or household supplies;
a family connect option, which when selected presents the user with a list of family members and a variety of ways in which to contact them;
a family legacy function, which when selected allows the user to record, store or publish recordings, videos or pictures, and make them accessible to family members and others;
and a community connect function, which when selected provides the user with several options for social or community-based communications or interaction.

14. The system of claim 13, wherein the community connect function comprises a connection with a social networking webpage, a senior communication, a religious or faith-based community, or an exercise group.

15. The system of claim 1, wherein the computing device is connected to a network or the Internet, and is monitored from a remote call or administrative center.

16. The system of claim 1, wherein computing device is located in the home or residence of the user.

17. The system of claim 16, wherein the user is a senior user.

18. The system of claim 1, wherein information from the computing device may be accessed by family members of the user who are located remotely from the home or residence of the user.
